(19) United States
(12) Patent Application Publication
(10) Pub. No.: US 2003/0041514 A1
(43) Pub. Date: Mar. 6, 2003

(54) Bagged tree multi-mode spray process

(76) Inventor: Leland F. Hazeltine, Woodland, CA (US)

Correspondence Address: Mark C. Jacobs, Esq., 3033 El Camino Avenue, Sacramento, CA, 95821, US

(21) Appl. No.: 09/945,224

(22) Filed: Sep. 4, 2001

(51) Int. Cl. A01B 79/02; A01G 1/00; A01C 1/00; A01H 3/00; A01B 79/00

(52) U.S. Cl. 47/58.1 SC

(57) ABSTRACT

A process for the eradication of undesirable plants on a plot of ground upon which young desired species are planted is disclosed. The desired trees or seedlings are covered with a sleeve that is preferably retained in place and then a spraying of the area with herbicide by land or air-based spray equipment takes place. Spraying may be from land or air-based spray equipment. This is followed by a removal of the sleeves from the trees and disposal thereof.

### FIELD OF THE INVENTION

This application relates to selective herbicidal treatment of defined plots of land by using various modes of application, but with tree protection.

### BACKGROUND OF THE INVENTION

In order to commercially propagate softwood forests such as pine, cedar and fir it is necessary to eradicate the brush, weeds, grasses and other tree varieties; all of which are classified collectively as undesirables because they compete for the water and sunlight of the forest. But, the trick is to eradicate the undesirables without damaging the “desired species” usually, softwoods of tree farms (cedar, pine, fir). In the process of the eradication of undesirables worker safety must be considered as well as effective use of herbicide to keep the cost of eradication to a minimum while achieving a high percentage of success. Sometimes, however, the desired specie is a hardwood such as oak or maple.

Accordingly, the process of this invention achieves the desired eradication with a high degree of success while providing protection of the “desired species”.

It is a first object to provide a new mode of protecting trees prior to the eradication of undesirables.

It is a second object to provide a plurality of modes for the application of herbicide to protected species of young trees.

It is a third object to provide an ecologically sound mode of protecting desired species during the eradication of undesirables.

It is a fourth object to provide a mode of eradication of undesirables that takes into account worker safety.

These and other objects of the invention will in part be obvious and will in part appear hereinafter.

The invention accordingly comprises the process possessing the features properties, and the relation of steps which are exemplified in the following detailed disclosure and the scope of the application of which will be indicated in the appended claims.

For a fuller understanding of the nature and objects of the invention reference should be made to the following detailed description taken in conjunction with the accompanying drawings.

The herbicide application process can be carried out in several modes such as, workers marching through a field, mechanical spraying by tractors, and spraying from helicopters and airplanes. The mode encompasses the application of herbicide in a forest wherein the young desired trees have been covered with paper sleeves and the sleeves optionally retained in place such that the undesired species of plants are killed off with little or no loss of the covered crop of a desired specie(s). The desired crop may be softwood (pine, fir, spruce, etc.) or hardwood (oak, maple, etc.).

While paper sleeves that are breathable are the preferred covering, a breathable plastic that will permit air to pass therethrough but not the larger molecules of herbicide, may also be employed. Paper is the preferred material because it is biodegradable.

### BRIEF DESCRIPTION OF THE FIGURES

FIG. 1 is typical softwood young tree such as, pine or fir.

FIG. 2 is a typical paper sleeve suitable for use in this process.

FIG. 3 is the paper sleeve of FIG. 2 in the process of being lowered over the young tree of FIG. 1.

FIG. 4 is a cutaway view of the tree of FIG. 1 disposed in the sleeve of FIG. 2 and the sleeve being retained in place.

FIG. 5 is a typical herbicide applicator person using the spraying technique of this invention for forest management.

FIG. 6 is a perspective view of the application of herbicide by plane to a field of protected trees.

FIG. 7 is a rear perspective view of the spraying of a field of protected trees by tractor mounted sprayers.

FIG. 8 is a perspective view of a truncated cone sleeve with bottom wings for soil retention.

FIG. 9 is a perspective view showing a pair of trees post spraying with sleeves post removal having been crumpled and discarded.

FIG. 10 is a diagrammatic view illustrating the formation of a cylindrical sleeve from flat paper stock.

### SUMMARY OF THE INVENTION

A process for forest management that permits a high degree of eradication of undesirables while protecting the desired species from the herbicide used for eradication. The process includes covering and, if necessary, retaining a breathable sleeve in position over a young tree while spraying the plot of ground having the covered young trees in one of several modes at ground level as well as from the air.

### DESCRIPTION OF THE PREFERRED EMBODIMENT

In FIG. 1 there is shown a typical young seedling tree age one to three years. Typical species to be propagated include many of the soft commercial woods such as, spruce, pine, fir and cedar as well as hardwoods such as maple and oak. The generic tree 35 may be any of these desired species.

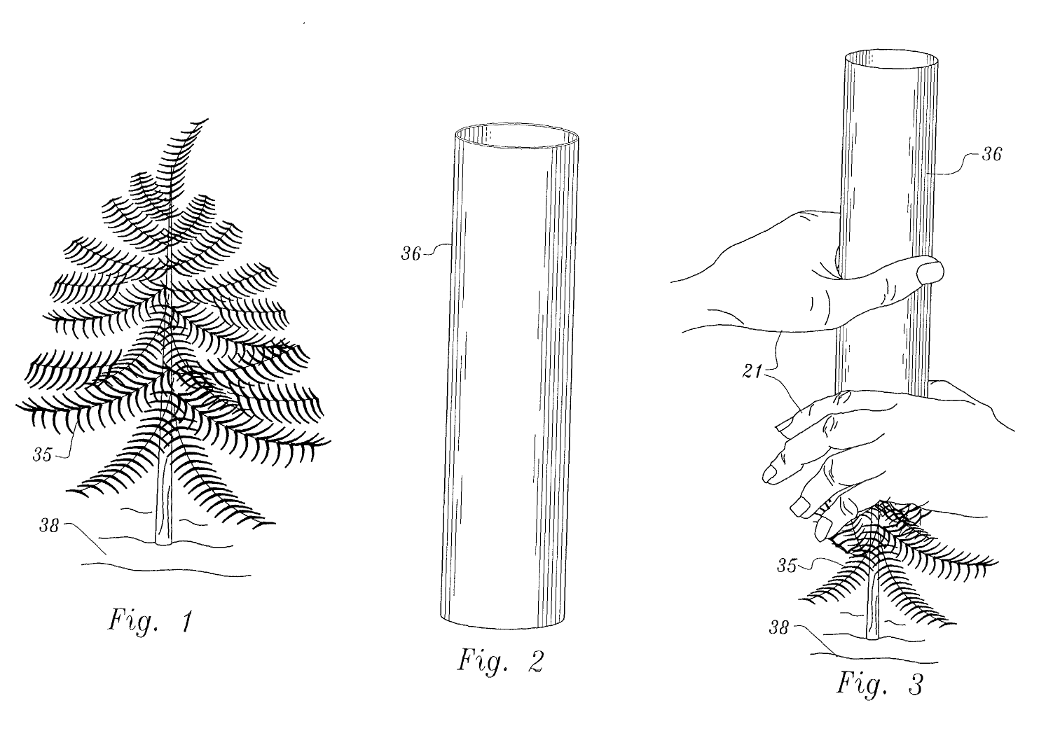

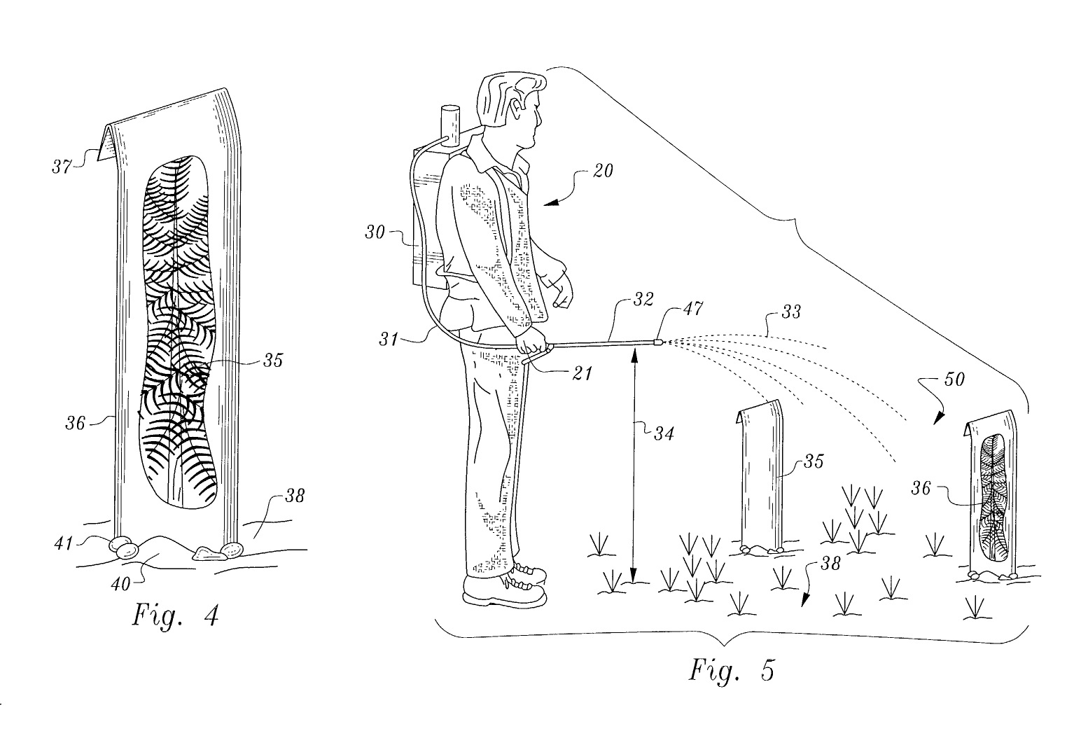

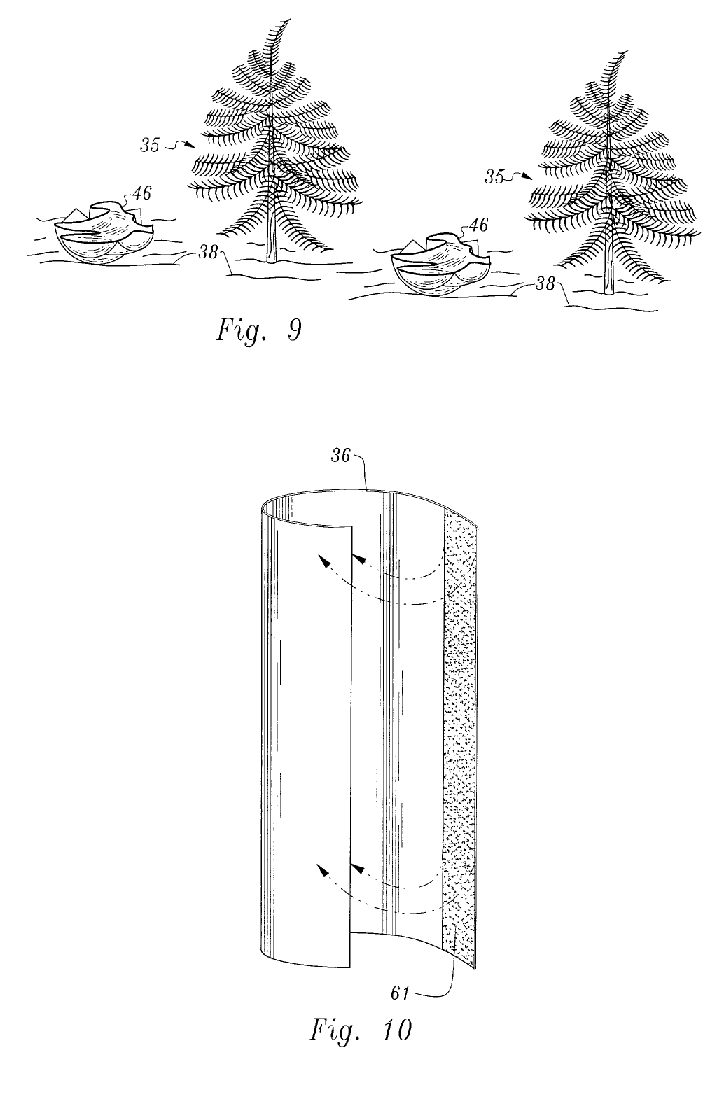

In FIG. 2 a typical paper sleeve or sack is shown and designated 36. The sleeve 36, which is breathable—air passes through it but not herbicide—is used to cover the tree 35 to prevent herbicide from contacting the desired species. Sleeves 36 which may include a flap 37 are made by various manufacturers. Flap 37 is formed by squeezing the top edge of the sleeve and bending the upper edge of sleeve 36 downwardly. In some instances it may be necessary to tear the sleeve slightly but in most instances such is not required. Most sleeves are formed, per FIG. 10, from flat stock which is then formed into a cylinder. The sleeve is formed of sufficient length such that the top—the area above the height of the tree—may be folded over to “seal” the tree body against any entry of chemical herbicides into the sleeve. It is this slight extra length of paper that forms flap 37 as seen in FIG. 4.

In FIG. 3 sleeve 36 is being lowered into position over tree 35 by the hands 21 of a worker. After the sleeve covers the tree, as in FIG. 4, the worker may fold over the top edge to form flap 37 to close off the sleeve top. He or she may optionally place some soil 40 around the base (bottom end) of the sleeve to retain the sleeve in position to prevent air from entering from the bottom of the sleeve thus keeping the sleeve from blowing up or off. Loose dirt 40 should be found within an area of approximately one to two feet in any direction on the earth floor 38 suitable for this purpose. Local sticks 41 of a size and weight suitable for the task may also be employed.

FIG. 4, as noted, is a cutaway view to show the tree in its protected environment, protected that is, from herbicide spray prior to spray application.

The process of this invention employs no specific spraying technique. A cadre of walking workers with wand sprayers is the mode of application of FIG. 5. Here worker 20, one of many employed for the project, carries a backpack sprayer 30 which has tubing 31 in fluid communication with a spray gun 32 which employs a wand with a trigger thereon which wand has preferably a particular nozzle 47 thereon and which is known to provide a fan like dispersal. The wand may be aimed high or carried at waist high elevation and when aimed horizontally results in a waterfall effect or arc type of spray. Such nozzles are available in the marketplace from several sources including Spray Systems, Inc. of Wheaton, Ill., among others. The 5500-Series Type X nozzle Sprayer from Spray Systems permits the spray stream to be adjusted to achieve the desired drop size and pattern to be employed according to this invention for ground workers. The worker 20 maintains his hand 21 having the spray wand 32 therein at about his or her own waist level. Arrows 34 shows aboveground height to be about thirty-four to thirty-six inches, depending on the height of the worker and retention of the spray wand 32. The workers line up in a straight line and move forward at the same rate of speed. As can be seen, the spray pattern 33 that emanates from the nozzle 47 is a waterfall shape pattern that falls in front of the feet of the worker 20.

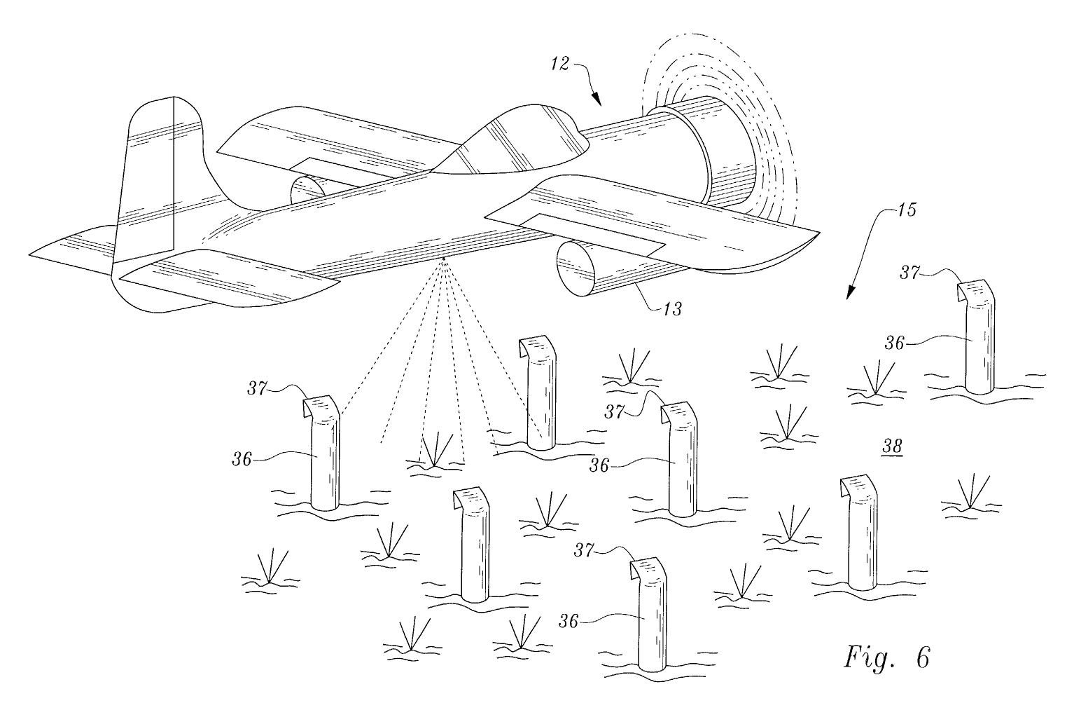

In FIG. 6 a field of sleeved trees 37 is being sprayed by a plane 12 having underwing mounted tanks 13. The mode of flying over the sleeve protected trees is conventional. The pilot flies in a manner similar to how he would fly during any herbicidal spray operation.

While a plane 12 is shown having two underwing spray tanks 13, a helicopter can be used just as effectively in this process to fly over field 15 with its sleeve 36 protected trees. Designator 38 signifies the ground level in which field 15 is situated.

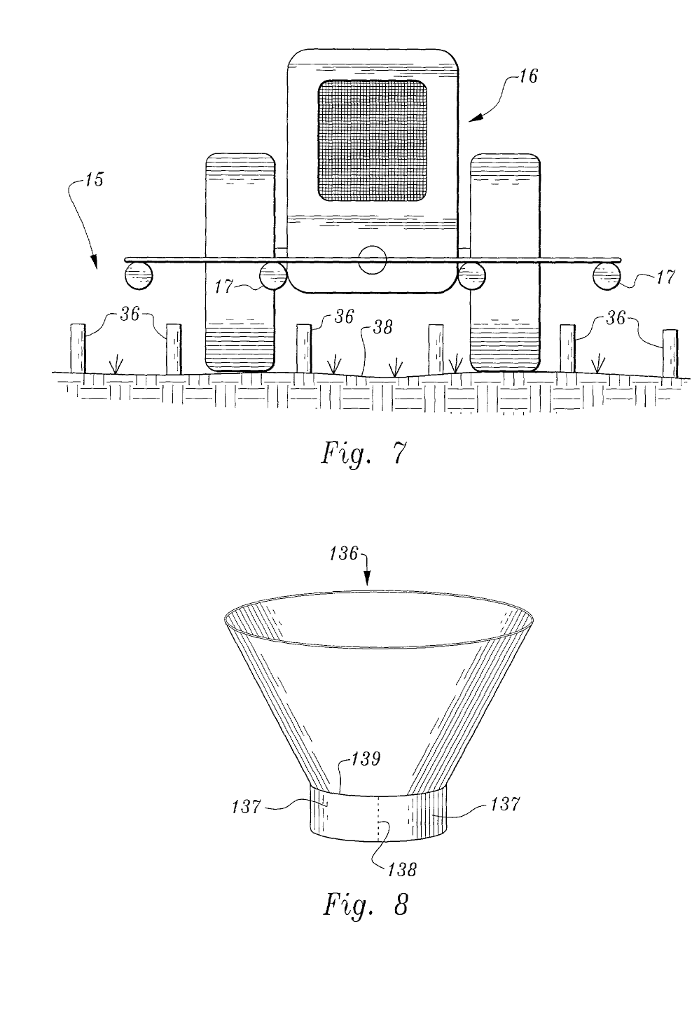

In FIG. 7 a tractor 16 is seen in a rear elevational view passing through a field 15. The tractor is seen at ground level 38. The tractor 16 has a series of boom mounted spray tanks 17 that spray the protected trees from an overhead position. The application procedure utilized is conventional but for the tree protection aspect.

Returning momentarily to the hand spraying mode, it is seen that worker safety is insured by spraying from the waist in a waterfall pattern as spray is aimed directly ahead and not at the applicator's face, eyes and body. If the movement of the hands of the plurality of workers is from a side to side motion, total coverage on each side of the worker's body will take place.

Adjustment of the spray distance from the focal point of the worker is something well within the skill of the art by a careful manipulation of the spray head trigger and by an artful choice of nozzle 47.

Nozzle choice is also within the skill of the artisan. If a usual amount of undesirables is at a location of a particular worker he or she can give that area an extra shot of herbicide and still not endanger fellow workers or upset the program.

Again, with reference to FIG. 5 one acceptable mode of walking worker application is known as the directed broadcast mode of application. In this procedure a cadre of workers walk in a single parallel line much like the soldiers of war in the U.S. Revolution or the war between the States. Each worker carries a backpack of herbicide and applies spray in an arc toward an area to be treated and away from the sleeve protected tree variety.

When and as the spraying procedure has been completed, with the addition of a day or two in case of high wind, workgroups can enter the treated zone and remove the individual sleeves 36, 136 of all types as discussed herein from the trees. The sleeves can be crumpled up as shown by designator 46 and left for collection or merely left on the earth to decompose.

Since most herbicides have a half-life, the efficacy of residual herbicide in the sleeves will be of little or no detriment to the environment. See FIG. 9.

It is seen that applicant has disclosed an improved process for eradicating undesirables while protecting the desired species from the impact of herbicide spray. The mode of application utilized herein assures total coverage of the land plot in question while simultaneously providing for worker safety.

While the discussion has centered on the removal of grasses, weeds and even hardwoods from commercial softwood tree farms, the process of this invention can also be used to eradicate undesirables from hardwood farms or forests as well.

The process is particularly beneficial to areas struck by fire where hundreds of seedlings are planted to reforest an area.

The utilizable sleeves herein may be of a paper or any biodegradable breathable material such as cotton or certain plastics. These materials will allow air in but not the much larger molecules of the herbicides. The process can be used with seedlings and young trees of the desired species.

While the use of clods of dirt is preferred as the retention member, other biodegradable or inert weights may be employed such as natural rocks or twigs.

If the desired specie seedling is wide relative to the height of the sleeve, in addition to forming a top flap workers may choose the sleeve of FIG. 8 which has a pair of openable wings at the bottom of the sleeve. See the FIG. 8 depicting same. Prior to separation, the wings may be retained by sand or pebbles in the nearby area. As can be seen from the drawings, the sleeves are generally cylindrical in configuration and vary in shape according to the tree to be protected. Such sleeves 136 may also be referred to as truncated cones with parallel ends. By selecting the preferred version, according to the general anatomy of the tree over which it is to be applied, a snug fit of the sleeve can be achieved. Thus, the choice of a cylinder or tapered cylinder will depend on the variety of tree to be protected from spray impaction.

While the primary purpose of this invention is for the protection of conifers and/or forests other trees, such as eucalyptus which is not a conifer, can be protected and treated, according to this invention, by using the tapered cone sleeve. Oaks and maples and other hardwood young trees may best be protected by the sleeve of FIG. 8 if they are the desired crop by use of the truncated coned sleeves.

It has been determined that the sleeves which are produced from overlapped stock of either a rectangular or trapezoidal configuration having a ½ inch overlap to form the particular cone can vary in size. The actual size to be used in any one particular job will vary according to the size of tree or seedling to be protected.

Cylindrical sleeve size can range from about a 6″ circumference with an elevation of about 10″ to a 10″ circumference X 20″, to a 12″ circumference X 24″ tall. {Circumference =ΠD} Truncated cones are preferably formed in the configuration wherein the top opening has a circumference of 1Z and the bottom has a circumference of 2Z. A typical example of the sizing of a tapered cylinder would have Z=2 for a 6.28″ circumference top with a 12.56″ lower circumference and a 12″ elevation.

Little or no discussion has been spent on the spray apparatuses used by walking workers who are spraying in the fields as these machines are deemed conventional. The backpack-mounted sprayer connects to the tubing leading to a spray wand which has a trigger actuation means thereon. The spray tip on the wand is the basis for determining the pattern of spray. This is seen to be analogous to a multi functional garden hose nozzle which permits a stream, mist, or fan shaped delivery pattern for water. A fan shape at waist height straight out will give rise to a waterfall pattern in both situations.

Tractor, helicopter & airplane mounted sprayers are also known to the art. The crux of the invention is therefore seen to be the placement of sleeve covers using a breathable paper or plastic sleeve over the trees prior to the spraying followed by removal of the sleeve covering post application. The employment of the top flap as well as the use of dirt 40 or other retainers around the bottom of the sleeve overlying a young tree to get a better sealing off of the tree inside from spray drops are also important aspects of this invention.

While tractors have been mentioned as one vehicle that can carry spraying equipment, other vehicles such as jeeps®, trucks and even trailers pulled by a motorized vehicle can carry spray equipment to do the job of applying herbicide.

Since certain changes may be made in the above process and device without departing from the scope of the invention herein involved, it is intended that all matter contained in the above description and shown in the accompanying drawings shall be interpreted as illustrative and not in a limiting sense.

I claim:

1. A process for the elimination of undesirables in favor of the encouragement of growth of selected desired species of trees which process comprises:
a. placing a sleeve over each desired seedling in a plot of land;
b. retaining the sleeve covering the desired specie member to prevent the sleeve from blowing away; and
c. spraying herbicide from a source thereof over the area of land to be treated.

2. The process of claim 1 wherein the spraying procedure is carried out by workers walking through the area to be treated with sprayers which have a spray wand held at waist level spray herbicide in a side to side motion in a waterfall pattern while walking from the point of entry onto the plot of ground to be treated to the point of exit of the plot of ground to be treated.

3. The process of claim 1 including the additional step of subsequently removing the sleeves from the desired species after the spraying is completed.

4. The process of claim 1 wherein the sleeve is of a truncated cone shape including the step of forming wings on each sleeve for the reception of weight to ensure the retention of the sleeve over the desired species.

5. The process of claim 1 including the step of forming a top flap on the sleeve to retard air currents from blowing the sleeve off the protected plant.

6. The process of claim 1 including the step of retaining the sleeve in place over the desired species by placing indigenous dirt, pebbles or twigs around the base of the sleeve.

7. The process of claim 1 wherein the spraying is carried out by a cadre of field workers spaced laterally apart in a straight line.

8. The process of claim 1 wherein the spraying is carried out by spraying from tanks mounted on an airplane or helicopter.

9. The process of claim 1 wherein the spraying is carried out by vehicle based spraying equipment.

10. The process of claim 6 further including the step of removal of the weights, removal of the sleeves, and crumbling of the sleeves and placement of the crumpled sleeves on the ground.

11. A process for eradicating undesirable tree species and protecting desirable tree species in a plot of ground from harm which comprises:
a. forming a flap on a paper sleeve and covering each member of the desired species, one sleeve per desirable tree;
b. retaining each sleeve by piling loose dirt around the base of the sleeve as it is placed over the desired specie;
c. forming workgroups of a finite number of member workers spaced a specified distance apart with
d. each worker applying herbicide in a waterfall pattern using a side to side motion; said workers spraying from the point of entry to the point of exit of the plot of ground to be treated; and
e. removing the sleeves from each desirable specie upon completion of the spraying.

12. The process of claim 11 further including crumpling each removed sleeve and casting it aside.

13. A process for the eradication of undesirable plants on a plot of ground upon which desired species are being propagated which process comprises:
a. covering each of the desired species with a truncated conical sleeve;
b. retaining the sleeve over the desired specie; and
c. spraying the plot of land containing the desired covered species with herbicide from sprayers mounted on a plane or helicopter.

14. The process of claim 14 wherein workers enter the sprayed field after the spraying and remove the sleeve from each tree.
